(19) United States
(12) Patent Application Publication
(10) Pub. No.: US 2021/0230636 A1
(43) Pub. Date: Jul. 29, 2021

(54) NUCLEIC ACID DELIVERY CARRIER, NUCLEIC ACID DELIVERY CARRIER SET, NUCLEIC ACID DELIVERY COMPOSITION, AND NUCLEIC ACID DELIVERY METHOD

(71) Applicant: KABUSHIKI KAISHA TOSHIBA, Tokyo (JP)

(72) Inventors: Eiichi AKAHOSHI, Shinagawa (JP); Mitsuko ISHIHARA, Setagaya (JP)

(73) Assignee: KABUSHIKI KAISHA TOSHIBA, Tokyo (JP)

(21) Appl. No.: 17/189,376

(22) Filed: Mar. 2, 2021

Related U.S. Application Data
(63) Continuation of application No. PCT/IB2020/051711, filed on Feb. 28, 2020.

(30) Foreign Application Priority Data
Jul. 23, 2019 (JP) 2019-135474

(51) Int. Cl. C12N 15/88; C12N 15/90; C12N 9/22; C12N 15/11; C12N 9/12

(52) U.S. Cl. CPC C12N 15/88; C12N 15/902; C12N 2310/20; C12N 15/11; C12N 9/1241; C12N 9/22

(57) ABSTRACT

According to one embodiment, a nucleic acid delivery carrier is used to integrate a first sequence into a genome of cells. The nucleic acid delivery carrier includes a donor DNA containing the first sequence, an RNA agent containing at least an RNA encoding a protein involving integration of the first sequence into the genome, and a lipid particle encapsulating the donor DNA and the RNA agent.

### CROSS-REFERENCE TO RELATED APPLICATIONS

This application is a Continuation Application of PCT Application No. PCT/IB2020/051711, filed Feb. 28, 2020 and based upon and claiming the benefit of priority from Japanese Patent Application No. 2019-135474, filed Jul. 23, 2019, the entire contents of all of which are incorporated herein by reference.

### FIELD

Embodiments described herein relate generally to a nucleic acid delivery carrier, a nucleic acid delivery carrier set, a nucleic acid delivery composition, and a nucleic acid delivery method.

### BACKGROUND

Recently, many functional proteins useful in genetic engineering have been discovered, including CRISPR-Associated Protein 9 (Cas9), which site-specifically cleaves DNA, and transposases, which excise a target DNA and insert it into cellular genome. To utilize such functional proteins in genetic engineering, a technology has been sought that can intracellularly introduce and express the functional proteins in a more efficient manner.

For instance, a method has been used, including delivery a functional protein-encoding DNA (e.g., a vector) into cells to express the functional protein intracellularly. Unfortunately, it is difficult for DNA alone to penetrate through a plasma membrane to enter cells. Examples of a method for delivery DNA into cells include a protocol using Lipofectamine. Lipofectamine can bind to nucleic acid to form a complex and makes it easy to introduce the nucleic acid into cells.

### BRIEF DESCRIPTION OF THE DRAWINGS

FIG. 1 is a cross-sectional view illustrating an example of nucleic acid delivery carrier according to an embodiment.

FIG. 2 is a flowchart showing an example of nucleic acid delivery method according to an embodiment.

FIG. 3 is cross-sectional views illustrating an example of nucleic acid delivery carriers according to embodiments.

FIG. 4 is cross-sectional views illustrating an example of nucleic acid delivery carrier set according to an embodiment.

FIG. 5 is a graph showing the experimental results of Example 1.

FIG. 6 is photomicrographs showing the experimental results of Example 1.

FIG. 7 is histograms showing the experimental results of Example 2.

FIG. 8 is histograms showing the experimental results of Example 3.

FIG. 9 is a graph showing the experimental results of Example 4.

FIG. 10 is histograms showing the experimental results of Example 4.

FIG. 11 is a graph showing the experimental results of Example 5.

FIG. 12 is electrophoresis images showing the experimental results of Example 6.

FIG. 13 is photomicrographs showing the experimental results of Example 6.

### DETAILED DESCRIPTION

In general, according to one embodiment, a nucleic acid delivery carrier according to an embodiment is used to integrate a first sequence into a genome of cells. The nucleic acid delivery carrier comprising: a donor DNA containing the first sequence; an RNA agent containing at least an RNA encoding a protein involving integration of the first sequence into the genome; and a lipid particle encapsulating the donor DNA and the RNA agent.

Various embodiments will be described hereinafter with reference to the accompanying drawings. Each drawing is a schematic diagram for facilitating embodiments and their understanding. In addition, drawings have sites where the form, size, and ratio differ from actual ones. These designs can be modified, if appropriate, while the following description and known technologies are taken into account.

A nucleic acid delivery carrier according to an embodiment includes a donor DNA containing a first sequence, an RNA agent containing at least an RNA encoding a protein involving integration of the first sequence into genome, and a lipid particle encapsulating the donor DNA and the RNA agent. This nucleic acid delivery carrier is used to integrate the first sequence into genome of cells (i.e., the first sequence into the cells). In addition, embodiments provide: a nucleic acid delivery carrier set including separate lipid particles, each encapsulating a donor DNA or an RNA agent; a nucleic acid delivery composition including the nucleic acid delivery carrier or the nucleic acid delivery carrier set; and a nucleic acid delivery method using the nucleic acid delivery carrier or the nucleic acid delivery carrier set. The following describes, in detail, the nucleic acid delivery carrier, the nucleic acid delivery carrier set, the nucleic acid delivery composition, and the nucleic acid delivery method.

### First Embodiment

Nucleic Acid Delivery Carrier

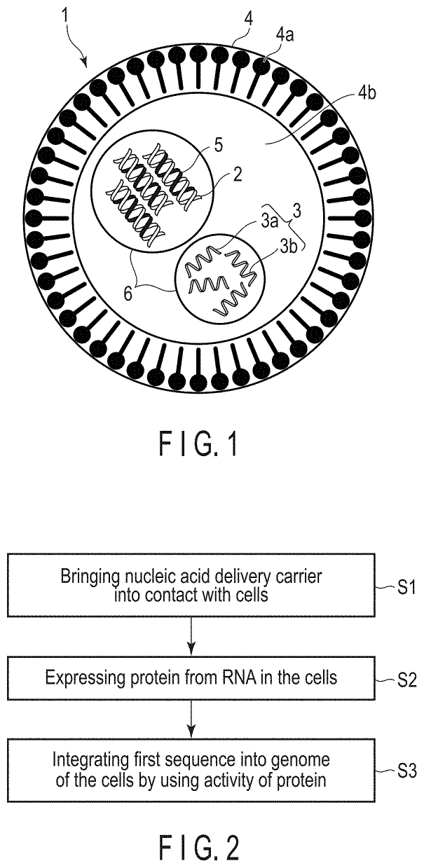

FIG. 1 is a cross-sectional view illustrating an example of nucleic acid delivery carrier according to the first embodiment. This nucleic acid delivery carrier 1 includes a donor DNA 2, an RNA agent 3, and a lipid particle 4 encapsulating the donor DNA 2 and the RNA agent 3. The donor DNA 2 includes a first sequence 5 to be integrated into genome of cells. The RNA agent 3 includes an RNA 3a and a guide RNA 3b. The RNA 3a is an RNA encoding a protein involving integration of the first sequence 5 into genome. The guide RNA 3b is an RNA containing a sequence corresponding to a genomic sequence into which the first sequence 5 is integrated (hereinafter, referred to as a “second sequence”). The donor DNA 2 and the RNA agent 3 are encapsulated in a state in which they are condensed using, for instance, a nucleic acid condensing peptide 6. The lipid particle 4 includes a lipid membrane produced by non-covalently aligning multiple lipid molecules 4a. The lipid particle 4 is an approximately spherical hollow body, which encapsulates the donor DNA 2 and the RNA agent 3 in its center cavity 4b.

Hereinafter, each element will be described in detail.

The donor DNA 2 is, for instance, a double-stranded linear DNA. The donor DNA 2 may be a single-strand DNA or a circular DNA. The length of the donor DNA 2 is, for instance, 3 to about 20000 nucleotides.

The first sequence 5 included in the donor DNA 2 is a sequence to be integrated into genome of cell and examples include: a gene expression cassette containing a promoter sequence, a specific gene, and a terminator sequence; a nucleotide sequence encoding a specific gene or part of the gene: or a naturally occurring nucleotide sequence or non-natural nucleotide sequence that is not a gene. The first sequence 5 may be a nucleotide sequence encoding one to several amino acids or a sequence composed of three to several dozen nucleotides. The length of the first sequence 5 is, for instance, 3 to about 20000 nucleotides.

For instance, the donor DNA 2 may contain, in addition to the first sequence 5, an additional sequence. Such a sequence may be a recognition sequence of a protein encoded by the RNA 3a or a recognition sequence of the guide RNA 3b.

How the donor DNA 2 is structured, that is, the kind of the first sequence 5, the kind of the additional sequence, the nucleotide length, and the like are selected depending on usage of the nucleic acid delivery carrier 1 and will be detailed later.

It is preferable that 1 to 100 molecules of the donor DNA 2 are included in the nucleic acid delivery carrier 1.

The RNA 3a is an RNA encoding a protein involving integration of the first sequence 5 into genome. This protein possesses activity such as DNA cleavage, joining, insertion and/or repair and is an enzyme involving integration of a DNA sequence into genome by using these activities. Hereinbelow, such a protein is also simply referred to as an “enzyme”. Examples of the preferable enzyme include: enzymes with endonuclease activity; transposases; reverse transcriptases and integrases (retroviral retrotransposases); reverse transcriptases and endonucleases (non-retroviral retrotransposases) and the like.

Examples of the enzyme with endonuclease activity include CRISPR-Associated Protein 9 (Cas9), zinc finger nuclease (ZEN), transcription activator-like effector nuclease (TALEN), meganuclease or the like. Each endonuclease involves integration of the first sequence 5 into genome by cleaving a phosphodiester bond where the first sequence 5 is integrated into genome as described in detail later.

Examples of the transposase include PiggyBac, SleepingBeauty, Frog Prince, Hsma, Minos, Tol1, Tol2, Passport, hAT, Ac/Ds, PIF, Harbinger, Harbinger3-DR, Himar1, Hermes, Tc3, Mos1 or the like. Each transposase has activity to excise the first sequence 5-containing sequence from the donor DNA 2 and to integrate it into genome, and thus involves the integration of the first sequence 5 into genome.

The RNA 3a may be an mRNA encoding, for instance, any of the above enzyme genes. The RNA 3a may have an additional sequence other than a sequence encoding the enzyme gene. Examples of the additional sequence include a 5′-end leader sequence, an IRES (Internal Ribosome Entry Site), a terminator sequence, or a poly (A) sequence. The RNA 3a may be capped.

The length of the RNA 3a is, for instance, about 20 to about 5000 nucleotides. It is preferable that 1 to about 1000 molecules of the RNA 3a are included in the nucleic acid delivery carrier 1. The RNA 3a may contain a plurality of RNAs encoding different kinds of enzyme.

The guide RNA 3b is an RNA having a nucleotide sequence corresponding to a second sequence or its complementary sequence. The second sequence is, for example, 15 to 25-mer sequence at or near the position where the first sequence 5 is introduced into genome of cells. The second sequence is a DNA and the guide RNA 3b is an RNA. Thus, the “corresponding nucleotide sequence” means a homologous nucleotide sequence or its complementary sequence except that T (thymine) of the second sequence is U (uridine) of the guide RNA 3b.

It is preferable to use the guide RNA 3b when the RNA 3a is an RNA encoding Cas9. At that time, the guide RNA 3b may be a guide RNA that can be designed, in the CRISPR-Cas9 system, based on the second sequence in accordance with common knowledge for those skilled in the art. In this case, the guide RNA 3b may be an RNA in which 3′ end-side crRNA containing a PAM sequence is ligated to the 3′ end of the second sequence or may be an RNA (sgRNA) in which a sequence including 3′ end-side crRNA containing a PAM sequence and part of tracrRNA is ligated to the 3′ end of the second sequence. The length of such a guide RNA 3b is, for instance, about 40 to about 150 nucleotides.

The guide RNA 3b is complexed with an endonuclease expressed from the RNA 3a and plays a role of guiding the endonuclease to the second sequence. Thus, use of the guide RNA 3b allows for site-specific integration of the first sequence 5. When the site-specific integration of the first sequence 5 is unnecessary or Cas9 is not used as the enzyme, the guide RNA 3b is not necessarily used.

It is preferable that 1 to about 1000 molecules of the guide RNA 3b are included in the nucleic acid delivery carrier 1.

The RNA agent 3 may include an additional RNA. Examples of the additional RNA include RNAs having a DNA-modifying function such as DNA methylation, demethylation, repair, and/or joining. For instance, these RNAs may each be an RNA encoding a protein having above modification activity. Inclusion of such an RNA makes it possible to add the modifications to the first sequence 5, which has been integrated into genome, and its surrounding sequence. Accordingly, for instance, the cell may be further functionally modified.

It is preferable that an RNA included in the RNA agent 3 may be modified to be resistant to degradation. For instance, the modification may be a known modification allowing the RNA not to be degraded by an intracellularly or extracellularly existing RNase. Such a modification, for instance, involves use/introduction of a naturally occurring modified nucleotide(s) or non-natural nucleotide(s) in the RNA, use/addition of a non-natural sequence(s) thereto, or addition of a naturally occurring/non-natural CAP structure thereto.

Examples of the naturally occurring modified nucleotide include pseudouridine, 5-methylcytidine, 1-methyl adenosine or the like. Examples of the non-natural nucleotide include BNA (Bridged Nucleic Acid), LNA (Locked Nucleic Acid), PNA (Peptide Nucleic Acid) or the like.

Examples of the non-natural sequence include an artificially synthesized, unnatural nucleotide sequence such as a random nucleotide sequence or a hybrid sequence made of nucleic acid and naturally occurring/non-natural amino acids. It is preferable that the non-natural sequence is added to, for instance, an end of RNA.

Examples of the naturally occurring CAP structure include CAP0 (m7GpppN), CAP1 (m7GpppNm) or the like. Examples of the non-natural CAP structure include ARCA (Anti-Reverse Cap Analog), LNA-guanosine or the like. It is preferable that the non-natural CAP structure is added to, for instance, the 5′ end of RNA.

Use of the RNA modified as above can prevent the RNA from degradation by an intracellularly or extracellularly existing RNase. This can result in an increased integration efficiency of the first sequence 5.

The nucleic acid condensing peptide 6 is for condensing many more nucleic acids into a small body to efficiently encapsulate the nucleic acids in the lipid particle 4. It is preferable to use, for instance, a cationic peptide as such a peptide. The cationic peptide can enter, for instance, a helical gap of anionic nucleic acid and shorten the gap to condense the nucleic acid.

The preferable nucleic acid condensing peptide 6 is, for instance, a peptide containing cationic amino acids in an amount of 45% or higher with respect to the total. The more preferable nucleic acid condensing peptide 6 has RRRRRR (the first amino acid sequence) on one end and a sequence RQRQR (the second amino acid sequence) on the other end. Further, 0 or more intermediate sequences consisting of RRRRRR or RQRQR are included between the above two amino acid sequences. In addition, two or more neutral amino acids are included between any two adjacent sequences of the first amino acid sequence, the second amino acid sequence, and the intermediate sequence. Examples of the neutral amino acid include G or Y.

The above nucleic acid condensing peptide 6 preferably has the following amino acid sequences:

(SEQ ID No. 1)
RQRQRYYRQRQRGGRRRRRR;
or
(SEQ ID No. 2)
RQRQRGGRRRRRR.

Such a nucleic acid condensing peptide can efficiently condense nucleic acid due to the cationic nature of R and can weaken the anionic property of the nucleic acid, thereby the nucleic acids are encapsulated in the lipid particle 4 more efficiently. Further, this nucleic acid condensing peptide can efficiently dissociate the nucleic acid in cells, thereby the nucleic acids introduced into the cell can be expressed efficiently in the cell.

Alternatively, the nucleic acid condensing peptide 6 has RRRRRR (the third amino acid sequence) on one end and has RRRRRR (the fourth amino acid sequence) on the other end. In addition, 0 or more intermediate sequences consisting of RRRRRR or RQRQR are included between the above two amino acid sequences. In addition, two or more neutral amino acids are included between any two adjacent sequences of the third amino acid sequence, the fourth amino acid sequence, and the intermediate sequence. Examples of the neutral amino acid include G or Y.

Such a nucleic acid condensing peptide 6 preferably has the following amino acid sequence:

(SEQ ID No. 3)
RRRRRRYYRQRQRGGRRRRRR.

Such a nucleic acid condensing peptide 6 has strong cationic nature at both ends and thus can efficiently bind to nucleic acid. Accordingly, the nucleic acids can be condensed more efficiently, thereby many more nucleic acids can be encapsulated in the lipid particle 4. This reduces the level of nucleic acid remaining outside the lipid particle 4, thereby preventing aggregation between the nucleic acid delivery carriers. Thus, each nucleic acid delivery carrier is likely to be incorporated into cells.

Further, the nucleic acid condensing peptide 6 having the following amino acid sequence may be used in combination with any of the above nucleic acid condensing peptides:

(M9)
(SEQ ID No. 4)
GNQSSNFGPMKGGNFGGRSSGPYGGGGQYFAKPRNQGGY.

This peptide can further condense an aggregated nucleic acid condensed by the above nucleic acid condensing peptide 6. Accordingly, a smaller size nucleic acid delivery carrier can be obtained. Such a nucleic acid delivery carrier is readily incorporated into cells, which makes integration of a nucleic acid into cellular genome more efficient.

Condensing of the donor DNA 2 and the RNA agent 3 can be carried out by, For instance, mixing and stirring the donor DNA 2 and the RNA agent 3 with the nucleic acid condensing peptide 6 before the encapsulation in the lipid particle 4. The donor DNA 2 and the RNA agent 3 may be together or separately condensed.

Since the above-described effects are exerted, it is preferable to use the nucleic acid condensing peptide 6. However, the nucleic acid condensing peptide 6 is not necessarily used depending on the kinds of the donor DNA 2 and the RNA agent 3 used or the kind of cell to be used.

The lipid particle 4 may be made of a lipid monolayer or a lipid bilayer. In addition, the lipid particle 4 may be made of a single layer membrane or a multi-layer membrane.

As a material for the lipid particle 4, for instance, lipids that are a main component of biological membrane can be used. Examples of such lipids include diacyl phosphatidylcholine, diacylphosphatidylethanolamine, ceramide, sphingomyelin, dihydrosphingomyelin, cephalin, and cerebroside. The case using diacyl phosphatidylcholine and diacylphosphatidylethanolamine is preferable, because the structure and the particle size of the lipid particle 4 is easy to control, and a membrane fusion potential can be imparted. It is preferable that the length of hydrocarbon chain of an acyl group included in the lipids is from C₁₀ to C₂₀. This hydrocarbon chain may be a saturated hydrocarbon group or an unsaturated hydrocarbon group.

Examples of the lipids that can be preferably used include: 1,2-dioleoyl-sn-glycero-3-phosphoethanolamine (DOPE), 1,2-stearoyl-sn-glycero-3-phosphoethanolamine (DSPE), 1,2-dipalmitoyl-sn-glycero-3-phosphatidylcholine (DPPC), 1-palmitoyl-2-oleoyl-sn-glycero-3-phosphatidylcholine (POPC), 1,2-di-O-octadecyl-3-trimethylammonium propane (DOTMA), 1,2-dioleoyl-3-dimethylammonium propane (DODAP), 1,2-dimyristoyl-3-dimethylammonium propane (14:0 DAP), 1,2-dipalmitoyl-3-dimethylammonium propane (16:0 DAP), 1,2-distearoyl-3-dimethylammonium propane (18:0 DAP), N-(4-carboxylbenzyl)-N,N-dimethyl-2,3-bis(oleoyloxy)propane (DOBAQ), 1,2-dioleoyl-3-trimethylammonium propane (DOTAP), 1,2-dioleoyl-sn-glycero-3-phosphochlorin (DOPC), 1,2-dilinoleoyl-sn-glycero-3-phosphochlorin (DLPC), 1,2-dioleoyl-sn-glycero-3-phospho-L-serine (DOPS), or cholesterol.

They have function to form the lipid particle 4 as well as have an increased effect of plasma membrane fusion and/or endocytosis when delivery the nucleic acid delivery carrier into cells.

The lipid particle 4 may be composed of a single lipid, but is preferably a lipid mixture including a plurality of kinds of lipids. The kind of lipid used for the lipid particle 4 is suitably selected while the size of the subject lipid particle 4, the kind of encapsulated material, the stability in a introduced cell, or the like are taken into consideration.

In addition to the above lipid, the lipid particle 4 preferably includes a first biodegradable lipid compound. The first biodegradable lipid compound may be represented by a formula:

Q-CHR2

wherein
Q is an oxygen-free nitrogen-containing aliphatic group containing two or more tertiary nitrogen atoms
R is, each independently, a C₁₂ to C₂₄ aliphatic group, and
at least one R contains, in a main chain or side chain thereof, a linker LR selected from the group consisting of —C(═O)—O—, —O—C(═O)—, —O—C(═O)—O—, —S—C(═O)—, —C(═O)—S—, —C(═O)—NH—, and —NHC(═O)—.

When the lipid particle 4 contains the first biodegradable lipid compound, the surface of the lipid particle 4 is non-cationic. Consequently, difficulty in the introduction into cell are decreased, so that nucleic acid delivery efficiency can be increased. As a result, it is possible to efficiently integrate the first sequence 5 into cellular genome.

Because of better nucleic acid encapsulation amount and nucleic acid delivery efficiency, it is preferable to use, as the first biodegradable lipid compound, for instance, a lipid with a structure represented by the following formulas.

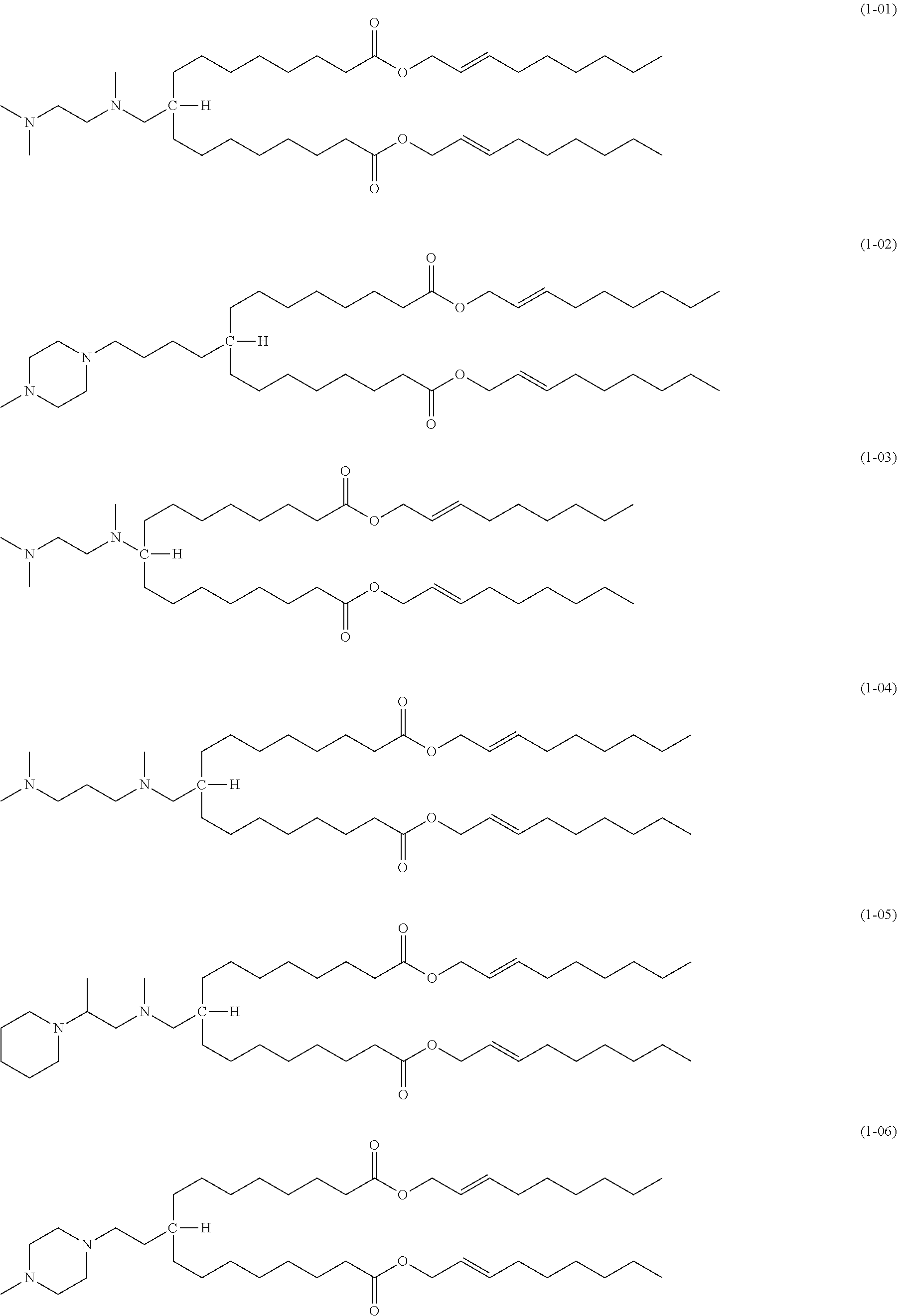

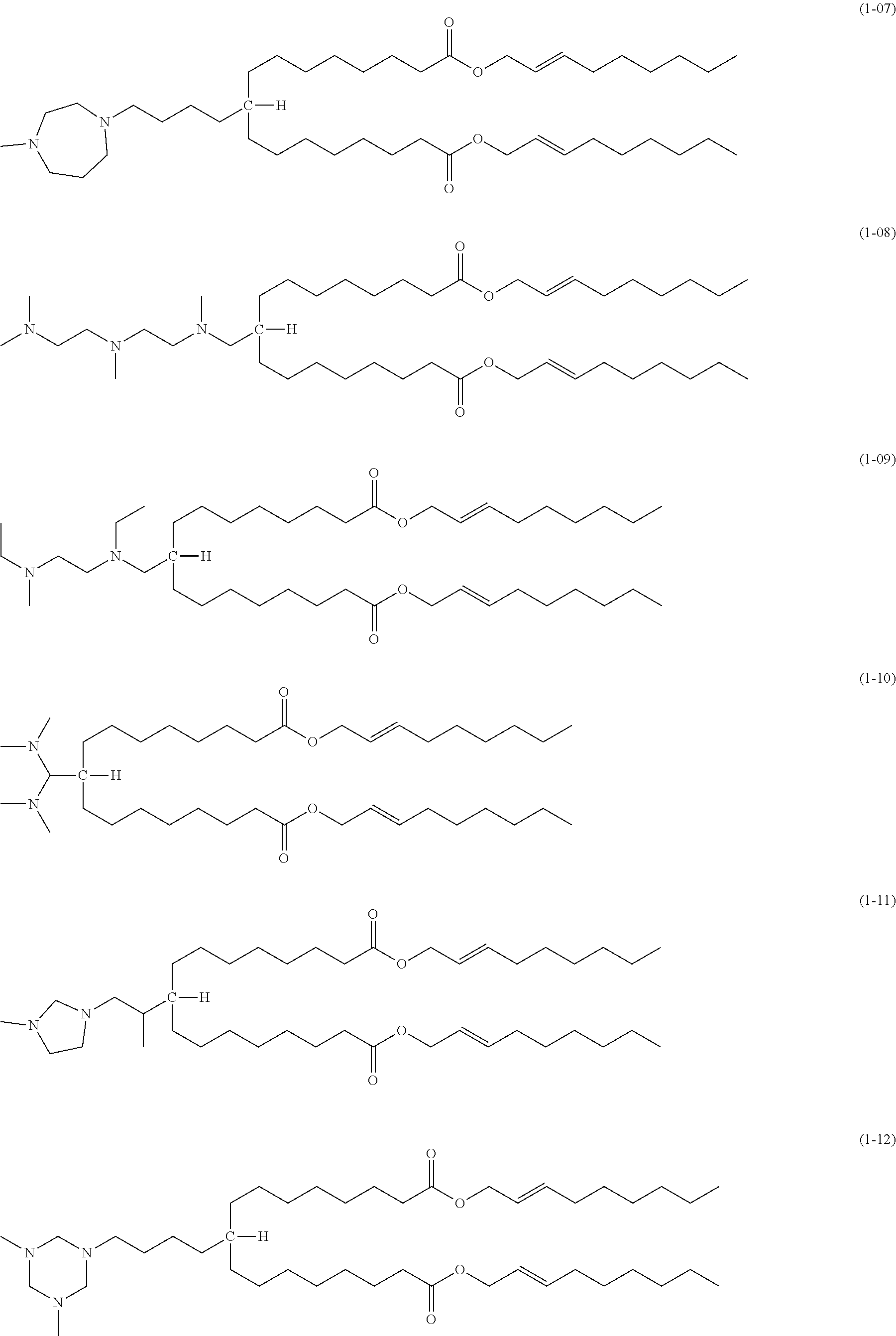

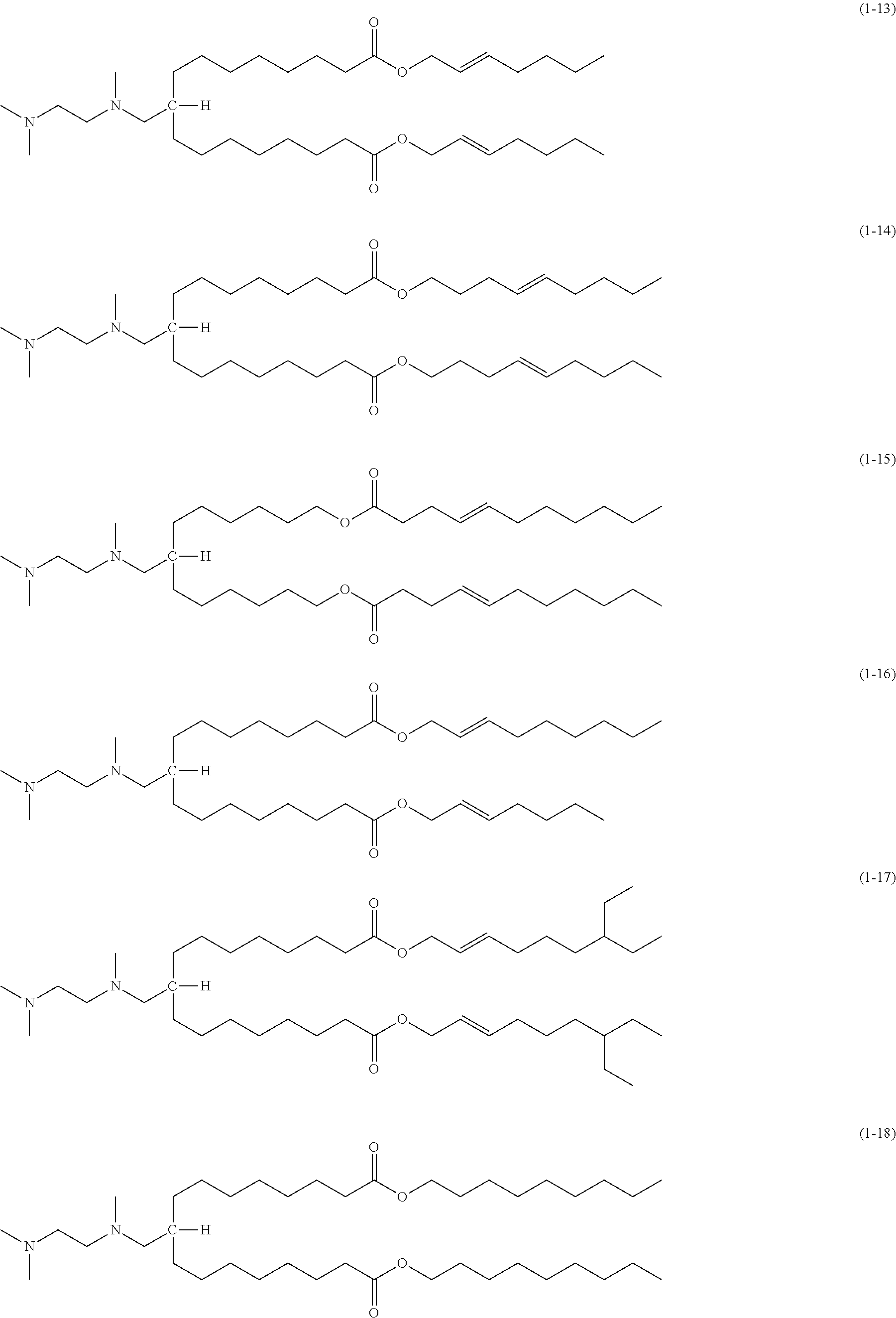

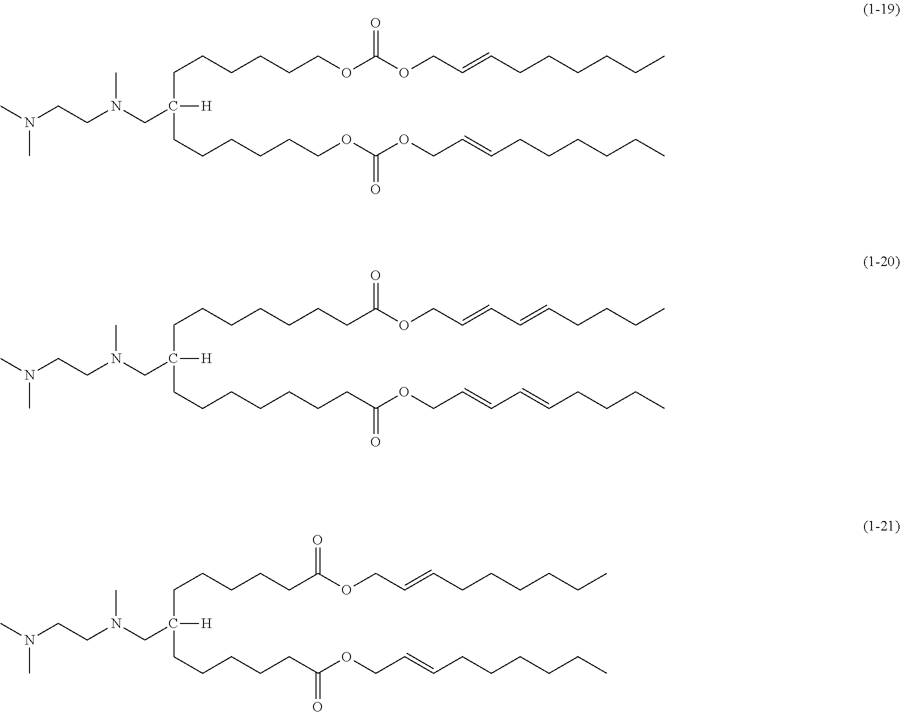

In addition, the lipid particle 4 preferably further includes, for instance, a second biodegradable lipid compound. The second biodegradable lipid compound may be represented by a formula:

P—[X—W—Y—W′—Z]₂

wherein
P is alkyleneoxy containing at least one ether bond in a main chain,
X is, each independently, a divalent linker containing a tertiary amine structure,
W is, each independently, C₁ to C₆ alkylene,
Y is, each independently, a divalent linker selected from the group consisting of a single bond, an ether bond, a carboxylic acid ester bond, a thiocarboxylic acid ester bond, a thioester bond, an amide bond, a carbamate bond, and a urea bond,
W′ is, each independently, a single bond or C₁ to C₆ alkylene, and
Z is, each independently, a fat-soluble vitamin residue, a sterol residue, or a C₁₂ to C₂₂ aliphatic hydrocarbon group.

In the case of including the second biodegradable lipid compound, the nucleic acid, etc., encapsulation amount may thus increase, since a hydrogen bond can be formed between an oxygen atom constituting an ether bond included in P and encapsulated nucleic acid.

Because of better nucleic acid encapsulation amount and nucleic acid delivery efficiency, it is preferable to use, for instance, the second biodegradable lipid compound having the following structures.

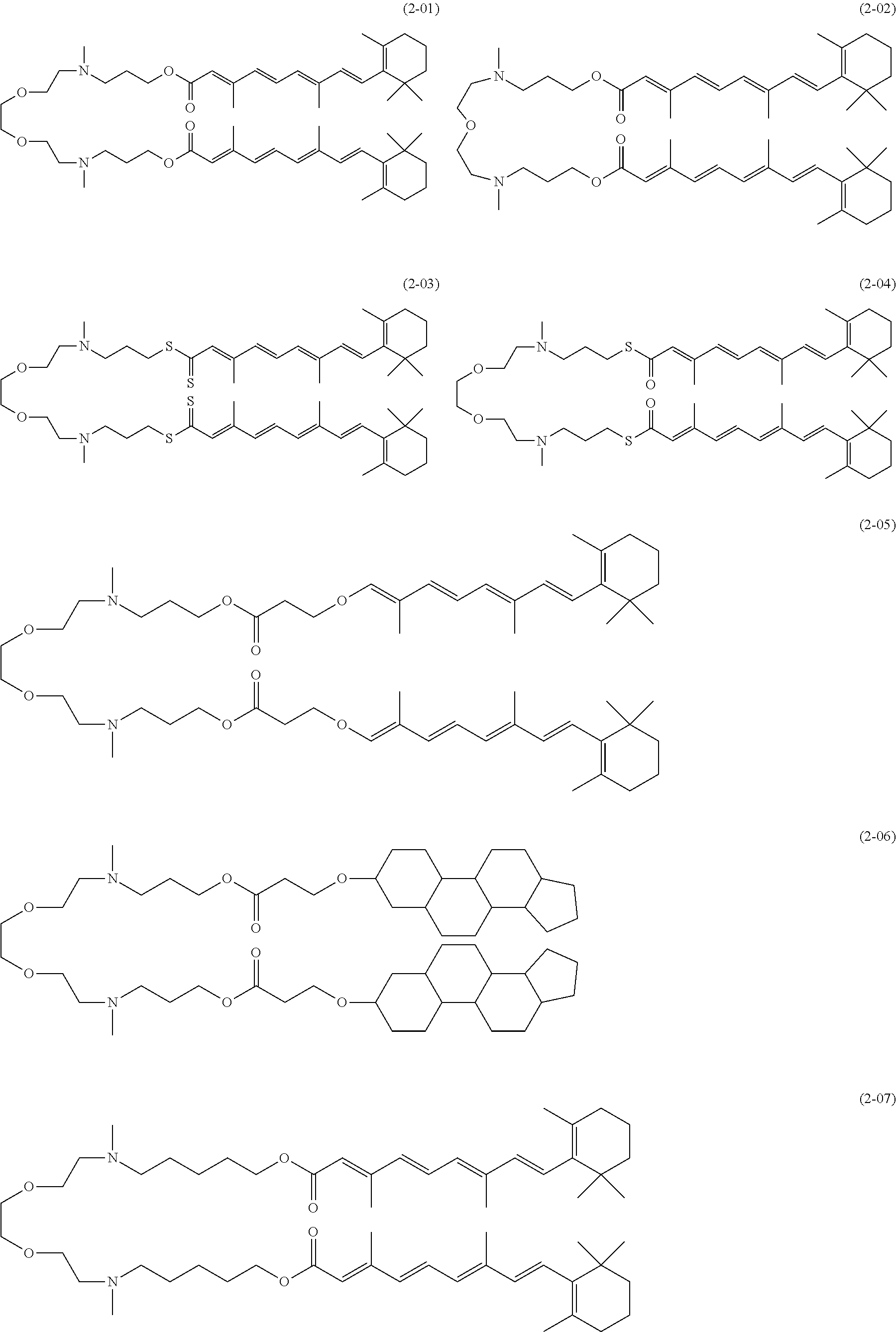

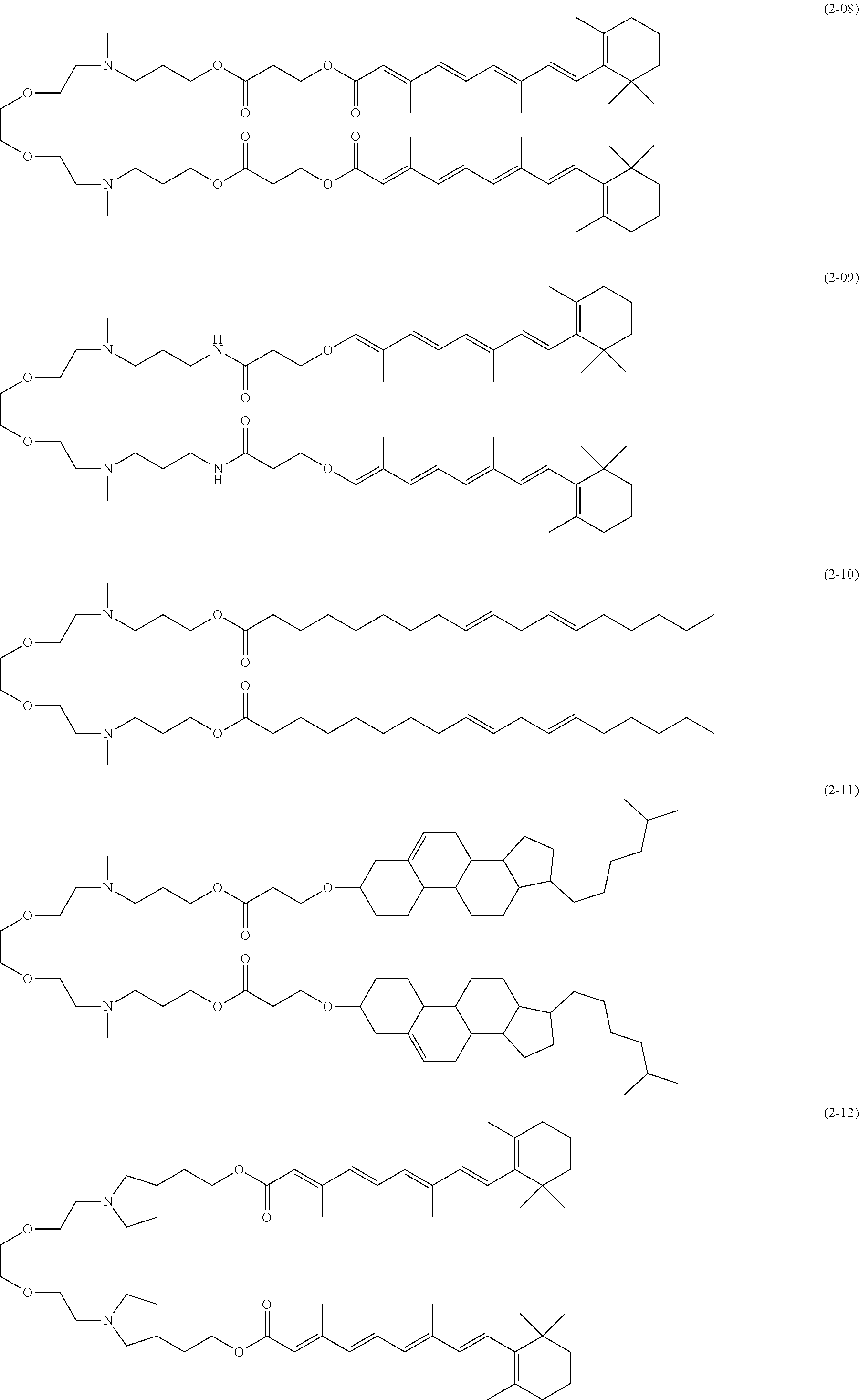

In the case of using the lipid particle 4 including the first and second biodegradable lipid compounds described above, the nucleic acid delivery efficiency is improved and cell death of the transfected cells can be reduced. In the cases where both the first biodegradable lipid compound and the second biodegradable lipid compound are included, they are readily applicable to gene therapy, nucleic acid medicine, genome diagnostics, and so on. It is particularly preferable to use the compound represented by formula (1-01) or formula (1-02) and the compound represented by formula (2-01), because of particularly excellent nucleic acid encapsulation amount and nucleic acid delivery efficiency.

The lipid particle 4 may further contain an additional lipid. Such an additional lipid may be optionally selected from those commonly used in the lipid particle. Examples of the additional material include: lipids that reduce aggregation between the lipid particles 4, such as polyethylene glycol (PEG)-modified lipids, in particular, polyethylene glycol (PEG) dimyristoyl glycerol (DMG-PEG), amino(oligoethylene glycol)alkanoic acid monomer-derived polyamide oligomer (U.S. Pat. No. 6,320,017 B), monosialoganglioside; toxicity-adjusting lipids with relatively low toxicity; lipids containing a functional group for binding a ligand to the lipid particle 4; or lipids for preventing leakage of encapsulated material such as sterol (e.g., cholesterol).

The case where the lipid particle 4 contains, for instance, the compound represented by formula (1-01) or formula (1-02), the compound represented by formula (2-01), DOPE and/or DOTAP, cholesterol, and DMG-PEG is preferable because of particularly excellent nucleic acid encapsulation amount and nucleic acid delivery efficiency. For instance, it is preferable that these components are included at any of compositions 1 to 6 listed in the following Table 1.

TABLE 1
Composition of Lipid Particle (Molar Ratio)

| | Compound of | Compound of | Compound of | | | | |
|---|---|---|---|---|---|---|---|
| | formula | formula | formula | | | | DMG- |
| | (1-01) | (1-02) | (2-01) | DOPE | DOTAP | Cholesterol | PEG |
| 1 | 73 | 0 | 0 | 44 | 0 | 59 | 4 |
| 2 | 73 | 0 | 0 | 0 | 44 | 59 | 4 |
| 3 | 73 | 0 | 0 | 22 | 22 | 59 | 4 |
| 4 | 73 | 0 | 30 | 44 | 0 | 59 | 4 |
| 5 | 73 | 0 | 30 | 0 | 44 | 59 | 4 |
| 6 | 73 | 0 | 30 | 22 | 22 | 59 | 4 |
| 7 | 0 | 73 | 0 | 44 | 0 | 59 | 4 |
| 8 | 0 | 73 | 0 | 0 | 44 | 59 | 4 |
| 9 | 0 | 73 | 0 | 22 | 22 | 59 | 4 |
| 10 | 0 | 73 | 30 | 44 | 0 | 59 | 4 |
| 11 | 0 | 73 | 30 | 0 | 44 | 59 | 4 |
| 12 | 0 | 73 | 30 | 22 | 22 | 59 | 4 |

The lipid particle 4 may encapsulate an additional compound in addition to the donor DNA 2 and the RNA agent 3. Examples of such a compound include: intracellular nucleic acid expression-regulating compounds such as retinoic acid, cyclic adenosine monophosphate (cAMP), or ascorbic acid; and therapeutic agents such as peptides, polypeptides, cytokines, growth factors, apoptosis factors, differentiation-inducing factors, cell surface receptors and their ligands, anti-inflammatory compounds, antidepressants, stimulant drugs, analgesics, antibiotics, contraceptive pills, antipyretics, vasodilators, angiogenic inhibitors, vasoactive agonists, signal transduction inhibitors, cardiovascular drugs, tumor medicines, hormones, and/or steroids.

The nucleic acid delivery carrier 1 may be manufactured by using, for instance, a known process used when encapsulating a small molecule in a lipid particle, etc., which process includes a Bangham method, organic solvent extraction, surfactant removal, freeze thaw or the like. For instance, an aqueous buffer containing the donor DNA 2 and the RNA agent 3 may be added to a mixture obtained by including a material for the lipid particle 4 in an organic solvent such as alcohol. The resulting mixture may be stirred and suspended to manufacture the nucleic acid delivery carrier 1. The volume ratio of the RNA agent 3 to the donor DNA 2 encapsulated in the lipid particle 4 may be easily adjusted by changing the volume ratio between the two in the aqueous buffer.

The encapsulation amounts of DNA and RNA may be determined by using, for instance, commercially available DNA and RNA quantification kits.

The nucleic acid delivery carrier 1 has an average particle size of from about 50 nm to about 300 nm and preferably from about 50 nm to about 200 nm. When the nucleic acid delivery carrier 1 is utilized for medical use, it is preferable that the nucleic acid delivery carrier 1 is a nano-order level particle. For instance, the particle size can be made smaller by ultrasonication. In addition, the size may be adjusted by making the nucleic acid delivery carrier 1 pass through a polycarbonate membrane or ceramic membrane. The average particle size of the nucleic acid delivery carrier 1 may be measured with a zetasizer by, for instance, dynamic light scattering.

Nucleic Acid Delivery Method

The following describes a nucleic acid delivery method using the above nucleic acid delivery carrier. The nucleic acid delivery method is a method for integrating the first sequence into cellular genome and includes bringing the nucleic acid delivery carrier into contact with cells.

FIG. 2 is a rough flowchart showing an example of the nucleic acid delivery method. The nucleic acid delivery method includes, for instance, the following steps:

(S1) bringing a nucleic acid delivery carrier into contact with cells;
(S2) through the contact, expressing a protein in the cells from an RNA included in an RNA agent; and
(S3) integrating a first sequence into genome of the cells by using activity of the protein.

In the nucleic acid delivery method, a performer of the method performs the step (S1), as a result of which the step (S2) and the step (S3) can spontaneously occur through the activity of molecule included in the nucleic acid delivery carrier and the intracellularly existing intrinsic mechanisms.

The cell may be derived from, for instance, a human, an animal, or a plant, or may be derived from a microorganism such as a bacterium or a fungus. The cell is preferably an animal cell, more preferably a mammalian cell, and most preferably a human cell. It is preferable that the cell is, for instance, a hematopoietic and immune cell, a mesenchymal cell, an epithelial cell, an endothelial cell, or a tissue stem cell or pluripotent stem cell.

The cell may be an ex vivo collected cell and may be, for instance, cell separated from body fluid such as blood or a tissue, or by biopsy. The cell may be, for instance, an isolated cell or a cell line. Alternatively, the cell may be an in vivo cell. The phrases “a cell”, “the cell” and “cells” may include both a cell (singular) and cells (plural, cell group, cell clump or cell cluster).

When the cell is an ex vivo collected cell or a microorganism, the step of bringing the nucleic acid delivery carrier 1 into contact with the cell 7 may include, for instance, adding the nucleic acid delivery carrier 1-containing composition onto the cell or microorganism cultured. For instance, it is preferable that after the addition, the cell is cultured for 30 to 48 h under conditions fit for cell survival.

If the cell is an in vivo animal cell, the contact is implemented by administering, in vivo, a composition containing the nucleic acid delivery carrier 1. The administration may be carried out through, for instance, a parenteral route by, for example, a subcutaneous, intravenous, intramuscular, intraarticular, intrasynovial, intrasternal, intraspinal, intraocular, intrahepatic, intralesional, or intracranial injection or infusion.

If the cell is a plant cell, the contact may be implemented by soaking the plant in the nucleic acid delivery carrier 1-containing composition or by injecting, using a syringe, etc., the composition into the plant.

The enzyme used in the nucleic acid delivery method of the embodiment is not limited to Cas9 and transposases, and the first sequence 5 can be likewise introduced by using an enzyme involving other nucleic acid transfer.

According to the nucleic acid delivery method of the embodiment, an enzyme may be introduced in an RNA form. Thus, a transcription step can be omitted when compared to the case of delivery in a DNA form, so that the enzyme can be expressed more rapidly and efficiently. Hence, the first sequence 5 can be integrated more efficiently.

In addition, if the enzyme in a protein form is introduced, it is necessary to adjust the size and composition of the lipid particle 4, depending on the size and characteristics of encapsulated protein. By contrast, if the RNA form is adopted like in the nucleic acid delivery method of the embodiment, the composition of the lipid particle 4 is relatively unrestricted. Thus, when compared to the case of delivery in a protein form, time and cost at the time of manufacture of the nucleic acid delivery carrier can be reduced.

In addition, when the enzyme in a DNA form is introduced, the enzyme gene can be integrated into cellular genome, and as a result of which an adverse effect may be exerted in the cell or in vivo tissue including the cell. By contrast, according to the nucleic acid delivery method of the embodiment, the enzyme is introduced in an RNA form. Consequently, the enzyme is not integrated into cellular genome and as a result of which an adverse effect can be prevented.

In addition, when nucleic acid is introduced into cells while conjugated with lipid such as Lipofectamine, the nucleic acid may be decomposed or aggregated with unwanted molecules. Also, it is difficult to adjust an abundance ratio of nucleic acid to be introduced. By contrast, according to the method of the embodiment, the donor 2 and the RNA agent 3 are encapsulated into the cavity 4b of the lipid particle 4. Thus, it is possible to protect the donor DNA 2 and the RNA agent 3 from degradation or aggregation. Further, it is possible to easily adjust the volume ratio between the donor DNA 2 and the RNA agent 3 to be introduced. Hence, the donor DNA 2 and the RNA agent 3 can be efficiently introduced into cells, the RNA can be expressed, and the first sequence 5 can then be integrated.

The delivery efficiency can be more increased by using the nucleic acid condensing peptide 6, by making the RNA resistant to degradation, and/or by including a biodegradable lipid compound in the lipid particle 4.

This nucleic acid delivery method is applicable to DNA transfection in, for instance, genome editing or gene recombination. For instance, when the first sequence 5 contains a gene expression cassette and a gene or a portion of gene, the gene may be integrated into cellular genome by the above nucleic acid delivery method. Thus, the cell can acquire the gene-mediated novel function. Or a normal function of the gene can be given to, for instance, the gene-deleted cell, the deficient cell, the gene defective cell, or the partially defective cell.

Alternatively, the first sequence 5 may be integrated to knockout a gene on cellular genome. For instance, a gene that expresses a product harmful to cells or a gene that overexpresses may be disrupted to give the cell a normal function. In addition, a gene knockout (KO) model organism can be created.

The nucleic acid delivery method is applicable to, without limitation, various fields such as gene therapy, model animal production, gene function analysis, drug discovery, gene drive, and/or genetically modified crop production. The method of the embodiment enables the target gene to be integrated more efficiently, thereby capable of more increasing gene therapy efficacy, model animal production efficiency, gene function analysis effectiveness, drug discovery efficiency, gene drive effectiveness or efficiency, or genetically modified organism production effectiveness or efficiency.

Composition

An embodiment provides a composition including the nucleic acid delivery carrier 1 and a vehicle.

Examples of the vehicle include water, saline such as physiological saline, an aqueous glycine solution, or a buffer.

The composition of the embodiment may include an additional component in addition to the nucleic acid delivery carrier and the vehicle. Examples of the additional component include, but are not limited to, stability-improving agents such as glycoproteins (e.g., albumin, lipoprotein, apolipoprotein, globulin); in the case of medical use, pharmaceutically acceptable determinants that make a pharmaceutical composition closer to physiological conditions, such as a pH modifier, a buffering agent, and a tonicity modifier (e.g., sodium acetate, sodium lactate, sodium chloride, potassium chloride, calcium chloride); lipid protector-like compounds that improve storage stability such as free radical-mediated damage-preventing lipophilic free radical quenchers (e.g., α-tocopherol) and lipid peroxidation damage-preventing water-soluble chelators (e.g., ferrioxamine). The vehicle and the additional component are preferably added after formation of the nucleic acid delivery carrier.

The composition may be, for instance, a pharmaceutical composition including components that can be administered pharmaceutically. In addition, the composition of the embodiment may be sterilized by a conventionally well-known procedure.

The composition may be provided as liquid or may be provided as dry powder. The powdery composition may be used by, for instance, dissolving it in a suitable liquid.

The concentration of nucleic acid delivery carrier included in the composition of the embodiment is not limited and is preferably from 0.01 to 30 mass % and more preferably from 0.05 to 10 mass %. The concentration is appropriately selected depending on the purpose.

Kit

An embodiment provides a kit including the nucleic acid delivery carrier. This kit contains, for instance, the above composition including a nucleic acid delivery carrier, and a reagent for delivery the nucleic acid delivery carrier into cells. Alternatively, it is possible to provide: a kit including separate containers, each containing a dispersion in which a material for the lipid particle 4 is dispersed in a vehicle, the donor DNA 2, and the RNA agent 3; and/or a kit including separate containers, each containing the dried lipid particle 4, the donor DNA 2, the RNA agent 3, and a vehicle. Further, it is possible to provide the dried lipid particle 4 or a dispersion containing a material for the lipid particle 4, and the donor DNA 2 and the RNA agent 3 as separate products and let a user choose each product depending on the purpose.

The kit may include, in another container, an additional chemical agent that can be included in the above composition.

### Second Embodiment

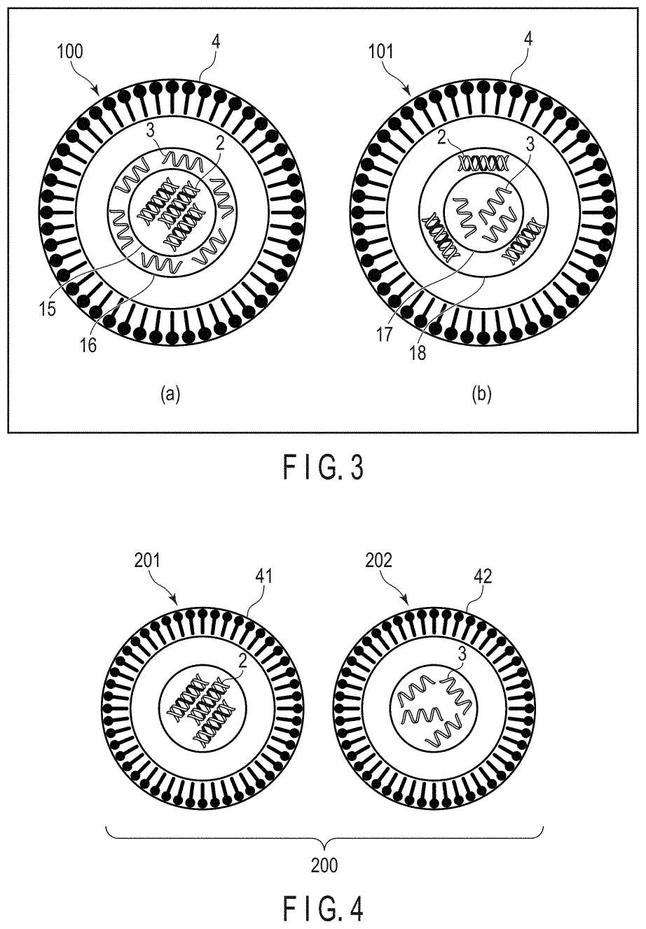

The second embodiment provides a nucleic acid delivery carrier in which a donor DNA 2 and an RNA agent 3 have a core-shell structure. FIG. 3 is cross-sectional views illustrating nucleic acid delivery carriers of the second embodiment.

The nucleic acid delivery carrier 100 shown in part (a) of FIG. 3 is provided with a core-shell structure including a donor DNA core 15 containing the donor DNA 2 and an RNA agent shell 16 that covers the donor DNA core 15 and contains the RNA agent 3. The core-shell structure is encapsulated in the lipid particle 4.

The nucleic acid delivery carrier 100, for instance, may be produced as follows. First, the donor DNA 2 is condensed using a nucleic acid condensing peptide to produce the donor DNA core 15. Next, the RNA agent 3 is made to contact the donor DNA core 15 and an RNA included in the RNA agent 3 is then electrostatically attached to the surrounding of the donor DNA core 15 to form the RNA agent shell 16. The RNA agent 3 may be condensed with a nucleic acid condensing peptide preliminarily. This results in formation of the core-shell structure. Subsequently, the core-shell structure is added to a solvent containing a material for the lipid particle 4. Then, the mixture is stirred, and the core-shell structure is thus encapsulated in the lipid particle 4. In this way, the nucleic acid delivery carrier 100 can be produced.

Such a structure allows for sequential delivery of the donor DNA 2 and the RNA agent 3. For instance, when the nucleic acid delivery carrier 100 is introduced into cells, the RNA agent 3 as a shell is released faster than the donor DNA 2 as a core. Then, an enzyme generated from an RNA included in the RNA agent 3 reaches the nucleus faster than the donor DNA 2. Due to this, the first sequence 5 integration starts as soon as the donor DNA 2 reaches the nucleus, so that the integration efficiency can be increased.

The nucleic acid delivery carrier 101 shown in part (b) of FIG. 3 is provided with a core-shell structure including an RNA agent core 17 containing the RNA agent 3 and a donor DNA shell 18 that covers the RNA agent core 17 and contains the donor DNA 2. The core-shell structure is encapsulated in the lipid particle 4.

For the nucleic acid delivery carrier 101, the RNA agent 3, for instance, is condensed using a nucleic acid condensing peptide to produce the RNA agent core 17 and the donor DNA 2 is made to contact the core. In this way, the donor DNA shell 18 is formed. The donor DNA 2 may be condensed with a nucleic acid condensing peptide preliminarily. Subsequently, the resulting core-shell structure is added to a solvent containing a material for the lipid particle 4. Then, the mixture may be stirred to produce the nucleic acid delivery carrier 101.

Because of having such a structure, when the nucleic acid delivery carrier 101, for instance, is introduced into cells, the donor DNA 2 as a shell is released faster than the RNA agent 3 and the RNA agent 3 is subject to sustained release. Due to this, even if an enzyme generated from the RNA agent is degraded in the cell, the enzyme can be supplied because the RNA is released again from the RNA agent core 17. Thus, the first sequence 5 integration effect can last for a long period of time.

The configuration of nucleic acid delivery carrier may be selected depending on the kind of cell used. For instance, in cells with slow protein degradation, that is, in cells which do not matter even if an enzyme generated from the RNA agent translocates into the nucleus faster, use of the nucleic acid delivery carrier 100 shown in part (a) of FIG. 3 makes it possible to increase the integration efficiency. Alternatively, for instance, in cells with faster cell cycle or in cells with rapid protein degradation, namely, in cells in which an enzyme is readily degraded or consumed, use of the nucleic acid delivery carrier 101 shown in part (b) of FIG. 3 makes it possible to increase the integration efficiency.

How fast nucleic acid included in the core is released and how long the release lasts may be adjusted by the composition or amount of nucleic acid condensing peptide or the amount of nucleic acid, etc.

The nucleic acid delivery carrier 100 or 101 may be used for a nucleic acid delivery method similarly to the nucleic acid delivery carrier of the first embodiment. In addition, each carrier may be provided as similar kit or composition as in the first embodiment.

### Third Embodiment

The third embodiment provides a nucleic acid delivery carrier set including separate lipid particles 4, each encapsulating a donor DNA 2 or an RNA agent 3. FIG. 4 is an example of the nucleic acid delivery carrier set. The nucleic acid delivery carrier set 200 includes a first carrier 201 and a second carrier 202. The first carrier 201 includes the donor DNA 2 and a first lipid particle 41 that encapsulates the donor DNA 2. The second carrier 202 includes the RNA agent 3 and a second lipid particle 42 that encapsulate the RNA agent 3. The donor DNA 2 or the RNA agent 3 is encapsulated in a state in which each is condensed using a nucleic acid condensing peptide.

The first carrier 201 and the second carrier 202 may be separately produced. For instance, the first carrier 201 or the second carrier 202 may be obtained by condensing either the donor DNA 2 or the RNA agent 3 by using a nucleic acid condensing peptide, and mixing and stirring it in a separate solution containing a material for each lipid particle.

The nucleic acid delivery carrier set 200 may be provided as a composition or a kit similar to the first embodiment. For instance, the first carrier 201 and the second carrier 202 are provided as compositions housed in, for instance, separate containers or compositions housed in the same single container.

The nucleic acid delivery carrier set 200 is applicable to a nucleic acid delivery method similarly to the nucleic acid delivery carrier of the first embodiment. According to such a nucleic acid delivery carrier set 200, either the first carrier 201 or the second carrier 202 may be made to first contact cells in the nucleic acid delivery method.

For instance, in the case of using above cell in which the RNA agent 3 is preferably first translocated into the nucleus, the second carrier 202 is preferably made to contact cells before the first carrier 201. For instance, it is preferable that at 30 min to 48 h after the second carrier 202 is in contact, the first carrier 201 is made to be in contact.

Alternatively, in the case of using cells in which the donor DNA 2 is first translocated into the nucleus and the RNA agent 3 is preferably subject to sustained release as described above, the first carrier 201 is preferably made to contact cells before the second carrier 202. For instance, it is preferable that at 30 min to 48 h after the first carrier 201 is in contact, the second carrier 202 is made to be in contact.

Optionally, both may be made to contact cells simultaneously.

When the donor DNA 2 and the RNA agent 3 are sequentially introduced by using the nucleic acid delivery carrier set 300, the delivery time difference is readily adjustable.

### EXAMPLES

The following describes examples of manufacture and use of the nucleic acid delivery carriers according to the embodiments.

### Example 1: Evaluation of Introduction Efficiency and Expression Efficiency of NanoLuc Gene by DNA-encapsulating Carrier

Preparation of DNA-Encapsulating Carrier

As the DNA, a plasmid DNA was used in which NanoLuc gene was ligated downstream of a cytomegalovirus promoter. To a DNA-solution containing this DNA, a cationic peptide was added to form a condensed DNA-peptide. Next, this was added to an ethanol-soluble fat solution (FFT10 (a biodegradable lipid compound represented by formula (1-01))/DOTAP/cholesterol/PEG-DMG=73/44/59/4 mol). Further, 10 mM HEPES (pH 7.3) was gently added. The mixture was then washed and enriched by centrifugal ultrafiltration to produce a DNA-encapsulating carrier. The DNA encapsulation amount of the carrier was measured with Quant-iT (registered trademark) PicoGreen dsDNA Assay Kit (manufactured by Thermo Fisher Scientific). Then, it was verified that the DNA was encapsulated in a sufficient amount.

Jurkat Preparation and Nucleic Acid Introduction Using DNA-encapsulating Carrier

Human T-cell leukemia cells (Jurkat, obtained from ATCC) was cultured in TexMACS medium (manufactured by Miltenyi Biotec K.K.). After the cells were recovered by centrifugation, the cells were suspended at 0.65×10⁷ cells in fresh TexMACS. Then, 150 μL of the cell suspension and TexMACS were added at 1.0×10⁶ cells/well onto a 48-well culture plate.

After that, the DNA-encapsulating carrier was added at 0.5 μg DNA/well to each well, and the mixture was cultured in an atmosphere at 37° C. and 5% CO₂.

Introduction of DNA into Cellsby Lipofectamine 3000

As a control, a Lipofectamine 3000 reagent (manufactured by Invitrogen) was used to introduce the above plasmid DNA into Jurkat. The introduction was carried out in accordance with the instructions attached to the reagent. The plasmid DNA was added at 0.5 μg/well to Jurkat and the mixture was cultured in an atmosphere at 37° C. and 5% CO₂.

Measurement of NanoLuc Expression Level (NanoLuc Luminescence Assay)

At 48 h after addition of the plasmid DNA mixed with the DNA-encapsulating carrier or Lipofectamine 3000, the culture plate was collected from an incubator. Then, a Nano-Glo Luciferase Assay System (manufactured by Promega) was used to measure the NanoLuc luminescence intensity by using a luminometer (Infinite (registered trademark) F200 PRO, manufactured by Tecan). The measurement was performed in accordance with the instructions attached to the kit and the device.

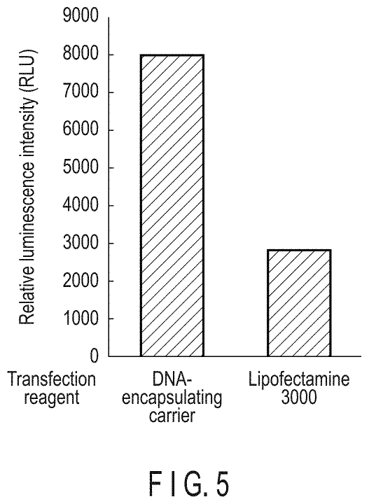

Results of NanoLuc Luminescence Assay FIG. 5 shows the results of measuring the NanoLuc luminescence intensity. The introduction using the DNA-encapsulating carrier caused a higher luminescence intensity than the case of introduction using Lipofectamine 3000. This result has demonstrated that in cells introduced with DNA by using the DNA-encapsulating carrier, the DNA is well introduced and the NanoLuc gene is well expressed. This indicates that the method of introduction with DNA encapsulated by the carrier has higher DNA introduction efficiency and gene expression efficiency than the method using a complex of DNA and Lipofectamine.

Microscopic Luminescent Cell Detection

Next, the cells introduced with DNA by using the carrier or Lipofectamine 3000 were used to detect luminescent cells by using a luminescence microscopy system (LV200, manufactured by OLYMPUS). At 24 h after the introduction, 100 μL of the cell culture liquid was transferred to a 4-well culture dish, and a NanoLuc substrate (Live Cell Luciferase Assay Kit, manufactured by Promega) was added. After the culture dish was set to a predetermined position in a luminescence microscopy system (LV200, manufactured by OLYMPUS), a light field image and a luminescent image of the cells were captured.

Microscopy Results

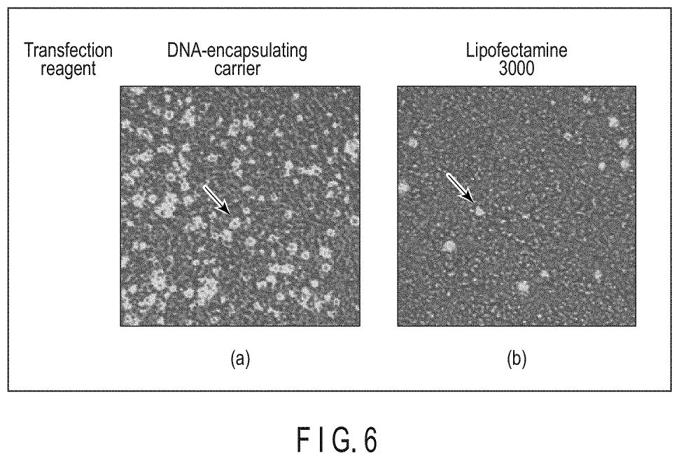

FIG. 6 shows captured images of the luminescent cells (images in which a light field image and a luminescent image were merged by Matamorph software). White dots indicated by the arrows in the photographs are luminescent cells. Part (a) of FIG. 6 shows a microscopic image of cells using the DNA-encapsulating carrier and part (b) of FIG. 6 is a microscopic image of cells using Lipofectamine 3000. The two were compared. It is evident that the case of using the DNA-encapsulating carrier had a much larger number of luminescent cells than the case of using Lipofectamine 3000. This result has demonstrated, like the NanoLuc luminescence assay, that the case of using the DNA-encapsulating carrier has higher DNA introduction efficiency and gene expression efficiency than the case of using a complex of Lipofectamine and DNA.

### Example 2: Evaluation of Introduction Efficiency and Expression Efficiency of Green Fluorescent Protein (GFP) Gene by mRNA-Encapsulating Carrier

Preparation of RNA-Encapsulating Carrier

As the messenger RNA (mRNA), green fluorescent protein (GFP) mRNA (manufactured by OZ Biosciences) as a reporter gene was used. An RNA solution containing this mRNA was added to an ethanol-soluble fat solution (FFT10/DOPE/cholesterol/PEG-DMG=73/44/59/4 mol) and the mixture was suspended by pipetting. Then, 10 mM HEPES (pH 7.3) was gently added. This solution was washed and enriched by centrifugal ultrafiltration to produce an RNA-encapsulating carrier. The RNA encapsulation amount of the carrier was measured with QuantiFluor (registered trademark) RNA System (manufactured by Promega), and it was verified that the mRNA was encapsulated in a sufficient amount.

Jurkat Preparation and Nucleic Acid Introduction Using RNA-encapsulating Carrier

Jurkat was cultured in TexMACS medium. After the cells were recovered by centrifugation, the cells were suspended at 0.65×10⁷ cells in fresh TexMACS. Then, 150 μL of the cell suspension and TexMACS were added at 1.0×10⁶ cells/well onto a 48-well culture plate.

After that, the RNA-encapsulating carrier was added at 0.5 μg mRNA/well to each well, and the mixture was cultured in an atmosphere at 37° C. and 5% CO₂.

Introduction of mRNA into Cells by Lipofectamine 3000

As a control, a Lipofectamine 3000 reagent was used to introduce the above mRNA into Jurkat. The introduction was carried out in accordance with the instructions attached to the reagent. The mRNA was added at 0.5 μg/well to Jurkat and the mixture was cultured in an atmosphere at 37° C. and 5% CO₂.

Detection of GFP Expression

At 24 h after addition of the mRNA mixed with the RNA-encapsulating carrier or Lipofectamine 3000, the culture plate was collected form an incubator. After recovered by centrifugation, the cells were suspended in phosphate buffer solution PBS containing 1% BSA (manufactured by Gibco, Thermo Fisher Scientific). Then, a fluorescence activated cell sorter (FACS; FACSVerse (registered trademark), manufactured by BD Biosciences) was used to detect green fluorescence of GFP.

Results

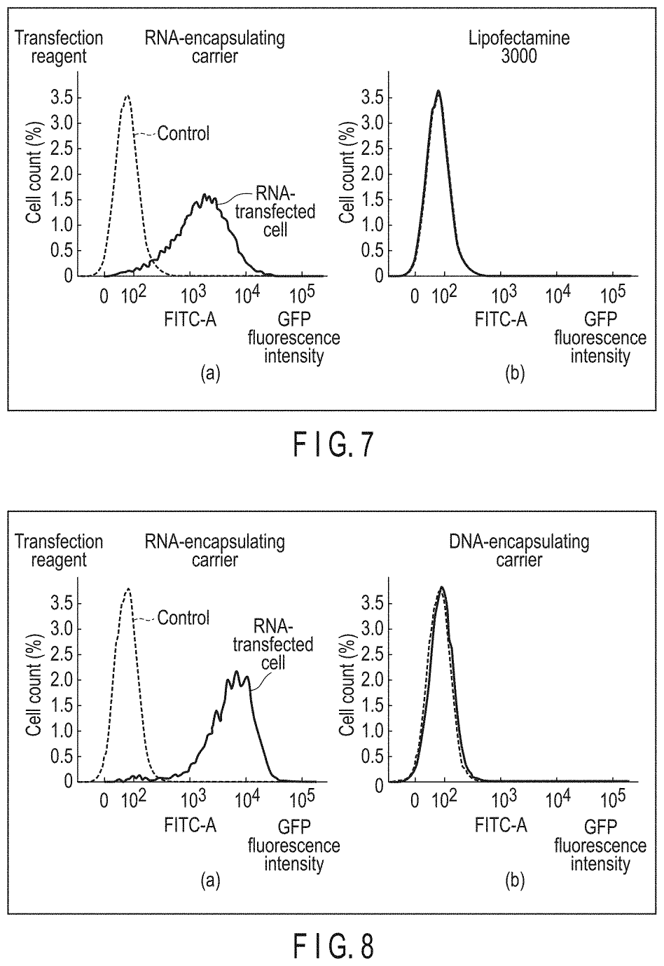

FIG. 7 shows the detection results. Part (a) of FIG. 7 shows the results of using the RNA-encapsulating carrier and part (b) of FIG. 7 shows the results of using Lipofectamine 3000. Each graph shows histograms in which the ordinate represents the cell count (%) and the abscissa represents the GFP expression intensity. Each solid line histogram shows the distribution of cells introduced with the RNA and each dashed line histogram shows the distribution of cells (control) introduced without RNA.

As illustrated in part (a) of FIG. 7, in the case of introduction with the RNA-encapsulating carrier, the distribution of fluorescence intensity of cells was significantly shifted to the right side when compared to that of control, suggesting that GFP is well expressed in the cells. This has revealed that the GFP mRNA was well introduced and GFP was well expressed.

By contrast, as illustrated in part (b) of FIG. 7, the distribution of fluorescence intensity in the case of introduction of RNA using a Lipofectamine reagent is almost identical to that of control, suggesting that the GFP mRNA introduction or expression is weak.

Hence, this indicates that the method of introduction with mRNA encapsulated by the carrier has higher mRNA introduction efficiency and gene expression efficiency than the method using a complex of mRNA and Lipofectamine.

### Example 3: Evaluation of GFP Gene Introduction Efficiency and Expression Efficiency by Introduction in mRNA Form (RNA-Encapsulating Carrier) and by Introduction in DNA Form (DNA-Encapsulating Carrier)

Preparation of RNA-Encapsulating Carrier

As the mRNA, the GFP mRNA described in Example 2 was used. An RNA solution containing the GFP mRNA was added to an ethanol-soluble fat solution (FFT10/DOPE/cholesterol/PEG-DMG=73/44/59/4 mol) and the mixture was suspended by pipetting. Then, 10 mM HEPES (pH 7.3) was gently added. This solution was washed and enriched by centrifugal ultrafiltration to produce an RNA-encapsulating carrier. The RNA encapsulation amount of the carrier was measured with QuantiFluor (registered trademark) RNA System, and it was verified that the mRNA was encapsulated in a sufficient amount.

Preparation of DNA-Encapsulating Carrier

As the DNA, a plasmid DNA was used in which GFP gene was ligated downstream of a cytomegalovirus promoter. Next, a cationic peptide was added to a DNA solution containing this DNA to condense the DNA. Then, this was added to an ethanol-soluble fat solution (FFT10/DOTAP/cholesterol/PEG-DMG=73/44/59/4 mol). Further, 10 mM HEPES (pH 7.3) was gently added. The mixture was then washed and enriched by centrifugal ultrafiltration to produce a DNA-encapsulating carrier. The DNA encapsulation amount of the carrier was measured with Quant-iT (registered trademark) PicoGreen dsDNA Assay Kit (manufactured by Thermo Fisher Scientific). Then, it was verified that the DNA was encapsulated in a sufficient amount.

Jurkat Preparation and Nucleic Acid Introduction Using Carrier

Jurkat was cultured in TexMACS medium. After the cells were recovered by centrifugation, the cells were suspended at 0.65×10⁷ cells in fresh TexMACS. Then, 150 μL of the cell suspension and TexMACS were added at 1.0×10⁶ cells/well onto a 48-well culture plate.

The RNA-encapsulating carrier or the DNA-encapsulating carrier was added at 1.0 μg/well to each well in separate well culture plates. Each plate was incubated in an atmosphere at 37° C. and 5% CO₂.

Detection of GFP Expression

At 24 h after addition of the carrier, each culture plate was collected from an incubator. After recovered by centrifugation, the cells were suspended in phosphate buffer solution (PBS) containing 1% BSA (manufactured by Gibco, Thermo Fisher Scientific). Then, FACS was used to detect green fluorescence (GFP) from the cells.

Results

FIG. 8 shows the detection results. Part (a) of FIG. 8 shows the results of using the RNA-encapsulating carrier and part (b) of FIG. 8 shows the results of using the DNA-encapsulating carrier. Each graph shows histograms in which the ordinate represents the cell count (%) and the abscissa represents the GFP expression intensity. Each solid line histogram shows the distribution of cells introduced with RNA or DNA using the corresponding carrier and each dashed line histogram shows the distribution of cells introduced without RNA or DNA.

As illustrated in part (a) of FIG. 8, the distribution of fluorescence intensity in the case of introduction of GFP gene in an mRNA form was significantly shifted to the right side when compared to that of control, suggesting that GFP is well expressed in the cells. This has revealed that the GFP mRNA was well introduced and GFP was well expressed.

By contrast, as illustrated in part (b) of FIG. 8, the distribution of fluorescence intensity in the case of introduction of GFP gene in a DNA form is almost identical to that of control, suggesting that the DNA introduction or GFP expression is weak.

Collectively, it has been demonstrated that the introduction of GFP in an mRNA form causes higher nucleic acid introduction efficiency and gene expression efficiency than the case of introduction in a DNA form.

### Example 4: Evaluation of GFP Gene Introduction Efficiency and Expression Efficiency by DNA/RNA-Co-Encapsulating Carrier

Preparation and Introduction of RNA/DNA-Co-Encapsulating Carrier into Jurkat

A mixed solution containing the NanoLuc gene-containing plasmid DNA described in Example 1 and the GFP gene-encoding mRNA described in Example 2 was added to an ethanol-soluble fat solution (FFT10/DOPE/cholesterol/PEG-DMG=73/44/59/4 mol). Further, 10 mM HEPES (pH 7.3) was gently added. The mixture was then washed and enriched by centrifugal ultrafiltration to produce an RNA/DNA-encapsulating carrier. The RNA encapsulation amount of the carrier was measured with QuantiFluor (registered trademark) RNA System and the DNA encapsulation amount was measured with Quant-iT (registered trademark) PicoGreen dsDNA Assay Kit. Then, it was verified that the mRNA and the DNA were encapsulated in sufficient amounts.

Jurkat was cultured in TexMACS medium. After the cells were recovered by centrifugation, the cells were suspended at 0.65×10⁷ cells in fresh TexMACS. Then, 150 μL of the cell suspension and TexMACS were added at 1.0×10⁶ cells/well onto a 48-well culture plate. After that, the DNA/RNA-encapsulating carrier was added at 0.5 μg mRNA and 0.5 μg DNA/well to each well, and the mixture was cultured in an atmosphere at 37° C. and 5% CO₂.

Introduction of mRNA and DNA into Cells by Lipofectamine 3000

As a control, a Lipofectamine 3000 reagent was used to introduce the mRNA and the plasmid DNA into Jurkat. The introduction was carried out in accordance with the instructions attached to the reagent. The mRNA and the plasmid DNA were each added at 0.5 μg/well to Jurkat and the mixture was cultured in an atmosphere at 37° C. and 5% CO₂.

Detection of NanoLuc and GFP Expression

Expression of NanoLuc from the NanoLuc DNA was detected with Nano-Glo Luciferase Assay System and expression of GFP from the GFP mRNA was detected by FACS. The detections were each conducted by a protocol described in Example 1 or 2.

Results

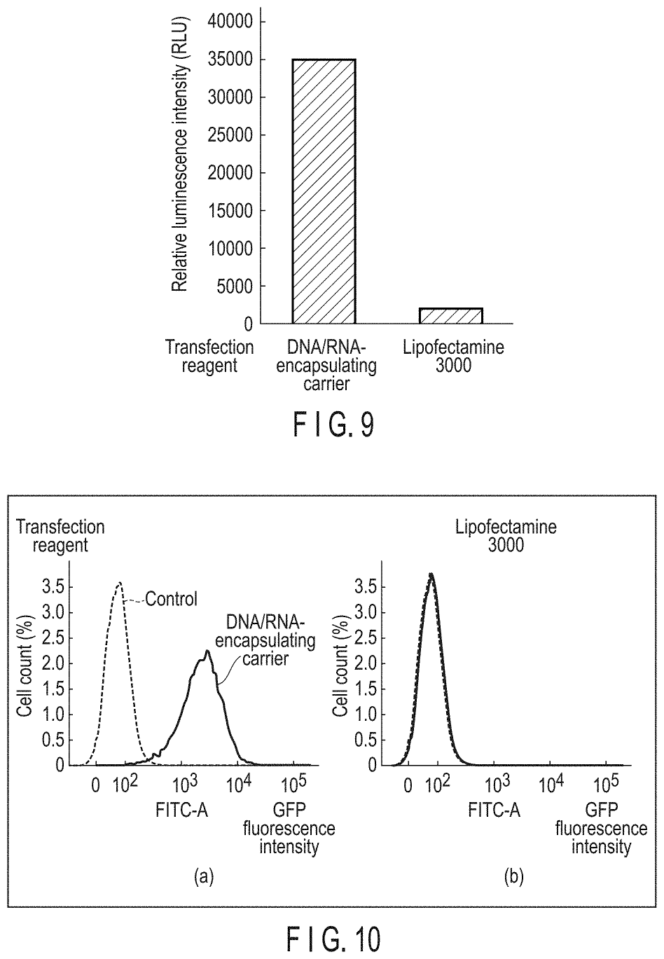

FIG. 9 shows the results of detecting the NanoLuc expression. The graph shown in FIG. 9 has revealed that the case of introduction of DNA and RNA using the carrier caused a much higher relative luminescence intensity than the case of using Lipofectamine 3000. This result indicates that the case of using the carrier has better DNA introduction efficiency and DNA-derived gene expression efficiency.

FIG. 10 shows the results of detecting the GFP expression. Part (a) of FIG. 10 shows the results of using the carrier and part (b) of FIG. 10 shows the results of using Lipofectamine 3000. These histograms have revealed that the case of using the carrier caused a higher GFP fluorescence intensity than the case of using Lipofectamine 3000. This result indicates that the case of using the carrier has better mRNA introduction efficiency and mRNA-derived gene expression efficiency.

The above results have demonstrated that the DNA/RNA-co-encapsulating carrier makes it possible to increase both DNA and RNA introduction efficiency and expression efficiency through simultaneous introduction.

### Example 5: Evaluation of Sequential Introduction of DNA and RNA by DNA-Encapsulating Carrier and RNA-Encapsulating Carrier

Preparation of DNA-Encapsulating Carrier

As the DNA, a plasmid DNA was used in which a NanoLuc gene expression cassette having a cytomegalovirus promoter and NanoLuc gene ligated was integrated. The DNA-encapsulating carrier was prepared by the protocol described in Example 1.

Preparation of RNA-Encapsulating Carrier

As the RNA, a transposase RNA was used. The RNA-encapsulating carrier was prepared by the protocol described in Example 2.

Cell Preparation and Nucleic Acid Introduction Using Carrier

Commercially available frozen human peripheral blood mononuclear cells (PBMC, Lonza) were thawed in a constant temperature incubator at 37° C., and the cells were recovered by centrifugation. The cells were suspended in TexMACS containing two different cytokines (10 ng/mL IL-7 and 5 ng/mL IL-15 (Miltenyi)), and were then seeded on a 6-cm culture dish. The cells were then cultured in an incubator under an atmosphere at 37° C. and 5% CO₂. After overnight culturing, the culture dish was collected from the incubator. The cells were recovered by centrifugation and suspended in TexMACS (containing 10 ng/mL IL-7 and 5 ng/mL IL-15), and were then cultured overnight in an atmosphere at 37° C. and 5% CO₂ on a 48-well culture plate coated with an anti-CD3 antibody (Miltenyi) and an anti-CD28 antibody (Miltenyi).

The transposase RNA-encapsulating carrier (4 μg) was added to the cell culture liquid, and the mixture was cultured in an atmosphere at 5% CO₂. After 2 h, the NanoLuc DNA-encapsulating carrier (4 μg) was further added, and the culturing was continued.

As a control, the transposase RNA-encapsulating carrier (4 μg) and the NanoLuc DNA-encapsulating carrier (4 μg) were simultaneously added to the same type cell culture liquid, and the mixture was cultured in an atmosphere at 5% CO₂.

Detection of NanoLuc Luminescence

At 48 h after addition of the first carrier, each culture plate was collected from an incubator. Then, a Nano-Glo Luciferase Assay System (manufactured by Promega) was used to measure each NanoLuc luminescence intensity by using a luminometer (Infinite (registered trademark) F200 PRO, manufactured by Tecan). Each luminescence was measured in accordance with the instructions attached to the kit and the device.

Results

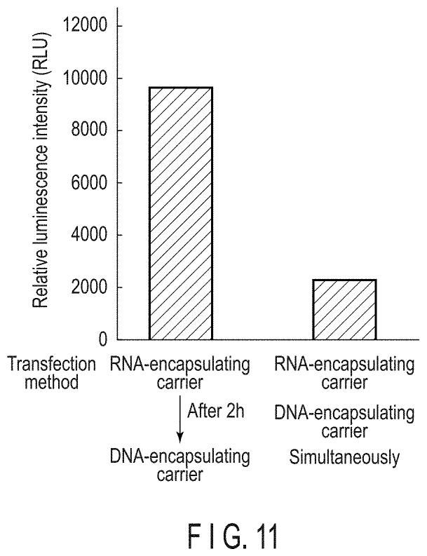

FIG. 11 shows the results of measuring the NanoLuc luminescence intensity. A higher NanoLuc luminescence intensity was detected in the case where the DNA-encapsulating carrier was added at 2 h after the RNA-encapsulating carrier was added than in the case where the DNA-encapsulating carrier and the RNA-encapsulating carrier were added simultaneously. This result has revealed that the sequential introduction of the mRNA of transposase assisting in DNA integration and the DNA containing a sequence to be integrated is effective in increasing the level of expression of a protein from the DNA. In addition, it has been demonstrated that the method of the embodiment makes it possible to efficiently introduce (transfect) and express a DNA even in PBMC that are generally considered to have low nucleic acid introduction efficiency.

### Example 6: Evaluation of Nucleic Acid Introduction Efficiency by Carrier Having DNA/RNA Core-Shell Structure

Preparation of DNA/RNA Core/Shell-Encapsulating Carrier

As the DNA, a plasmid DNA was used in which a CAR gene expression cassette having a cytomegalovirus promoter and CAR gene ligated was integrated, and as the RNA, the GFP mRNA described in Example 2 was used. A cationic peptide was added to a DNA solution containing this DNA to form a DNA core. Then, the above RNA was added to form an RNA shell around the DNA core. As a result, a solution containing the resulting DNA/RNA core/shell was prepared. Next, this was added to an ethanol-soluble fat solution (FFT10/DOTAP/cholesterol/PEG-DMG=73/44/59/4 mol). Further, 10 mM HEPES (pH 7.3) was gently added. The mixture was then washed and enriched by centrifugal ultrafiltration to produce a carrier having the DNA/RNA core/shell structure.

Preparation of DNA/RNA Mixture-Encapsulating Carrier

As a control, a cationic peptide was added to a mixed solution containing the above DNA and RNA to prepare a solution containing a DNA/RNA mixture core. This was added to an ethanol-soluble fat solution (FFT10/DOTAP/cholesterol/PEG-DMG=73/44/59/4 mol). Further, 10 mM HEPES (pH 7.3) was gently added. The mixture was then washed and enriched by centrifugal ultrafiltration to produce a DNA/RNA mixture-encapsulating carrier.

Examination of Encapsulation of DNA/RNA in Carrier

Next, to examine whether or not the DNA and the RNA were encapsulated in both the resulting carriers, each carrier was raptured (by adding a surfactant: sodium dodecyl sulfate) and each core structure was disintegrated (by adding polyglutamic acid). Then, each released DNA/RNA was detected by agarose electrophoresis.

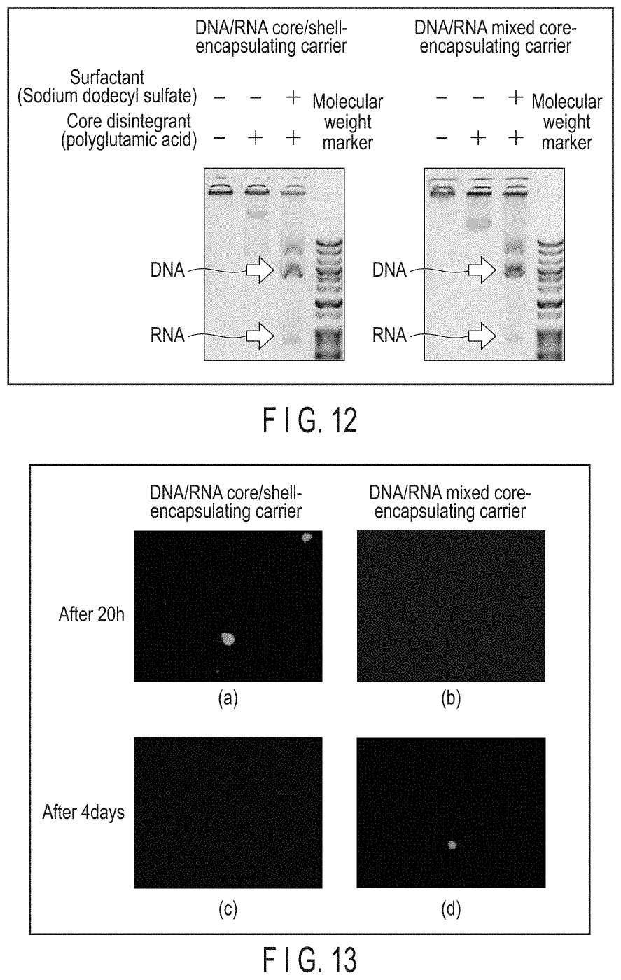

FIG. 12 shows the detection results. In both the DNA/RNA core/shell-encapsulating carrier and the DNA/RNA mixed core-encapsulating carrier, the DNA and RNA signals (arrowed in the images) were detected when both the carrier rapture and the core structure disintegration were conducted at one time. This result has demonstrated that both the DNA/RNA core/shell-encapsulating carrier and the DNA/RNA mixed core-encapsulating carrier contained DNA and RNA aggregates.

Cell Fluorescence Microscopy

At 20 h after Jurkat was introduced with either the DNA/RNA core/shell-encapsulating carrier or the DNA/RNA mixed core-encapsulating carrier, green fluorescence-emitting cells (GFP-expressing cells) were detected under a fluorescence microscope. FIG. 13 shows photomicrographs indicating the results. In the case of the DNA/RNA mixed core-encapsulating carrier, GFP-expressing cells were not detected at the time point of 20 h after the carrier delivery (part (b) of FIG. 13). However, a GFP-expressing cell(s) was detected after 4 days (part (d) of FIG. 13). By contrast, in the case of the DNA/RNA core/shell-encapsulating carrier, GFP-expressing cells were detected at 20 h after the carrier addition (part (a) of FIG. 13). These results suggest that expression of RNA in the DNA/RNA core/shell occurs faster than that in the DNA/RNA mixed core. In conclusion, it has been demonstrated that in the case of the DNA/RNA core/shell-encapsulating carrier, the DNA/RNA core/shell is sequentially disintegrated intracellularly and the corresponding proteins can be expressed in the order from the shell RNA to the core DNA.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

1. A nucleic acid delivery carrier suitable for the integration of a DNA sequence into a genome of cells, the nucleic acid delivery carrier comprising:
a donor DNA comprising the DNA sequence;
an RNA agent comprising an RNA encoding a protein capable of integrating the DNA sequence into the genome of cells; and
a lipid particle encapsulating the donor DNA and the RNA agent.

2. The carrier of claim 1, wherein the donor DNA and the RNA agent have a core-shell structure in which the donor DNA is a core and the RNA agent is a shell.

3. The carrier of claim 1, wherein the donor DNA and the RNA agent have a core-shell structure in which the RNA agent is a core and the donor DNA is a shell.

4. The carrier of claim 1, wherein the protein is an enzyme with endonuclease activity.

5. The carrier of claim 4, wherein the protein is CRISPR-Associated Protein 9 (Cas9).

6. The carrier of claim 5, wherein the RNA agent further comprises a guide RNA comprising a sequence corresponding to a sequence on the genome of cells into which said DNA sequence is integrated.

7. The carrier of claim 1, wherein the protein is a transposase.

8. The carrier of claim 7, wherein the protein is PiggyBac, SleepingBeauty, Frog Prince, Hsma, Minos, Tol1, Tol2, Passport, hAT, Ac/Ds, PIF, Harbinger, Harbinger3-DR, Himar1, Hermes, Tc3, or Mos1.

9. The carrier of claim 1, wherein the RNA agent further comprises an RNA encoding a DNA-methylating protein, an RNA encoding a DNA-demethylating protein, an RNA encoding a DNA repair protein, and/or an RNA encoding a DNA-binding protein.

10. The carrier of claim 1, wherein the RNA included in the RNA agent is modified to be resistant to degradation.

11. The carrier of claim 1, wherein the donor DNA and/or the RNA agent are condensed using a nucleic acid condensing peptide.

12. The carrier of claim 1, wherein the lipid particle further comprises a first biodegradable lipid represented by a formula:
Q-CHR₂
wherein
Q is an oxygen-free nitrogen-containing aliphatic group containing two or more tertiary nitrogen atoms,
R is, each independently, a C₁₂ to C₂₄ aliphatic group, and
at least one R contains, in a main chain or side chain thereof, a linker LR selected from the group consisting of —C(═O)—O—, —O—C(═O)—, —O—C(═O)—O—, —S—C(═O)—, —C(═O)—S—, —C(═O)—NH—, and —NHC(═O)—.

13. The carrier of claim 1, wherein the lipid particle further comprises a second biodegradable lipid represented by a formula:
P—[X—W—Y—W′—Z]₂
wherein
P is alkyleneoxy containing at least one ether bond in a main chain,
X is, each independently, a divalent linker containing a tertiary amine structure,
W is, each independently, C₁ to C₆ alkylene,
Y is, each independently, a divalent linker selected from the group consisting of a single bond, an ether bond, a carboxylic acid ester bond, a thiocarboxylic acid ester bond, a thioester bond, an amide bond, a carbamate bond, and a urea bond,
W′ is, each independently, a single bond or C₁ to C₆ alkylene, and
Z is, each independently, a fat-soluble vitamin residue, a sterol residue, or a C₁₂ to C₂₂ aliphatic hydrocarbon group.

14. A nucleic acid delivery carrier set suitable for the integration of a DNA sequence into a genome of cells, the nucleic acid delivery carrier set comprising:
a first carrier comprising a donor DNA comprising the DNA sequence and a first lipid particle encapsulating the donor DNA; and
a second carrier comprising an RNA agent comprising an RNA encoding a protein capable of integrating the DNA sequence into the genome of cells, and a second lipid particle encapsulating the RNA agent.

15. A nucleic acid delivery composition, comprising:
a vehicle; and
the carrier of claim 1 or a nucleic acid delivery carrier set suitable for the integration of a DNA sequence into a genome of cells, the nucleic acid delivery carrier set comprising:
a first carrier comprising a donor DNA comprising the DNA sequence and a first lipid particle encapsulating the donor DNA; and
a second carrier comprising an RNA agent comprising an RNA encoding a protein capable of integrating the DNA sequence into the genome of cells, and a second lipid particle encapsulating the RNA agent.

16. A nucleic acid delivery method for integrating a DNA sequence into a genome of cells by using a nucleic acid delivery carrier, the method comprising:
bringing the nucleic acid delivery carrier into contact with the cell,
wherein the nucleic acid delivery carrier comprises
a donor DNA comprising the DNA sequence,
an RNA agent comprising an RNA encoding a protein capable of integrating the DNA sequence into the genome of cells, and
a lipid particle encapsulating the donor DNA and the RNA.

17. The method of claim 16, further comprising:
bringing the nucleic acid delivery carrier into contact with the cells to express, in the cell, the protein from the RNA; and
integrating the DNA sequence into the genome of the cells by using activity of the protein.

18. The method of claim 16, wherein the donor DNA and the RNA agent in the nucleic acid delivery carrier have a core-shell structure in which the donor DNA is a core and the RNA agent is a shell.

19. The method of claim 18, wherein the protein expressed from the RNA reaches a nucleus of the cells faster than the donor DNA.

20. The method of claim 16, wherein the donor DNA and the RNA agent in the nucleic acid delivery carrier have a core-shell structure in which the RNA agent is a core and the donor DNA is a shell.

21. The method of claim 20, wherein the RNA is subject to sustained release in the cell.

22. A nucleic acid delivery method for integrating a DNA sequence into a genome of cells, the method comprising:
bringing the first carrier and the second carrier into contact with the cell,
wherein the first carrier comprises a donor DNA comprising the DNA sequence and a lipid particle encapsulating the donor DNA, and
wherein the second carrier comprises an RNA agent comprising an RNA encoding a protein capable of integrating the DNA sequence into the genome of cells and a lipid particle encapsulating the RNA agent.

23. The method of claim 22, wherein the first carrier is brought into contact with the cell, and then the second carrier is brought into contact with the cell.

24. The method of claim 22, wherein the second carrier is brought into contact with the cell, and then the first carrier is brought into contact the cell.

25. The method of claim 22, wherein the first carrier and the second carrier are simultaneously brought into contact with the cell.

26. The method of claim 16, wherein the protein is an enzyme with endonuclease activity.

27. The method of claim 26, wherein the protein is CRISPR-Associated Protein 9 (Cas9).

28. The method of claim 27, wherein the RNA agent further comprises a guide RNA comprising a sequence corresponding to a sequence on the genome of cells into which the DNA sequence is integrated.

29. The method of claim 16, wherein the protein is a transposase.

30. The method of claim 29, wherein the protein is PiggyBac, SleepingBeauty, Frog Prince, Hsma, Minos, Tol1, Tol2, Passport, hAT, Ac/Ds, P1F, Harbinger, Harbinger3-DR, Himar1, Hermes, Tc3, or Mos1.


SEQUENCE LISTING (4 sequences)

SEQ ID NO: 1 - protein, 20 aa, Artificial Sequence
  Nucleic acid condensing peptide
RQRQRYYRQRQRGGRRRRRR

SEQ ID NO: 2 - protein, 13 aa, Artificial Sequence
  Nucleic acid condensing peptide
RQRQRGGRRRRRR

SEQ ID NO: 3 - protein, 21 aa, Artificial Sequence
  Comperative example of nucleic acid condensing peptide
RRRRRRYYRQRQRGGRRRRRR

SEQ ID NO: 4 - protein, 39 aa, human
GNQSSNFGPMKGGNFGGRSSGPYGGGGQYFAKPRNQGGY